(19) United States
(12) Patent Application Publication
(10) Pub. No.: US 2016/0051560 A1
(43) Pub. Date: Feb. 25, 2016

(54) TREATMENTS INVOLVING ESLICARBAZEPINE OR ESLICARBAZEPINE ACETATE

(71) Applicant: BIAL - PORTELA & Cª, S.A., São Mamede do Coronado (PT)

(72) Inventor: Patricio Manuel Vieira Araújo SOARES DA SILVA, São Mamede do Coronado (PT)

(73) Assignee: BIAL- Portela & Ca, S.A., Sao Mamede do Coronado (PT)

(21) Appl. No.: 14/780,922

(22) PCT Filed: Apr. 4, 2014
(86) PCT No.: PCT/PT2014/000021

§ 371 Date: Sep. 28, 2015

(30) Foreign Application Priority Data
Apr. 4, 2013 (GB) 1306095.9

(51) Int. Cl. A61K 31/55; A61K 9/16; A61K 9/10; A61K 9/20

(52) U.S. Cl. CPC A61K 31/55; A61K 9/20; A61K 9/16; A61K 9/10

(57) ABSTRACT

The invention provides a drug selected from eslicarbazepine acetate and eslicarbazepine, for use in preventing the recurrence/relapse of Bipolar Disorder 1 and/or one or more episodes associated with Bipolar Disorder 1 in a human patient.

### FIELD OF THE INVENTION

The invention relates to new therapeutic techniques involving eslicarbazepine acetate or eslicarbazepine.

### BACKGROUND OF THE INVENTION

Bipolar disorder is a chronic, recurrent, severe, and often debilitating illness characterised by one or more episodes of mania, depression and long-term psychosocial disability. Bipolar disorders in general include bipolar disorder and unstable bipolar disorder with rapid fluctuations (rapid cyclers), manic-depressive disorders, acute mania, mood episodes, and manic and hypomanic episodes.

As defined in the Diagnostic and Statistical Manual of Mental Disorders, Fourth Edition (DSM-IV), Bipolar Disorder 1 is characterized by one or more manic or mixed mood episodes, usually accompanied by Major Depressive Episodes. Subsequent episodes (that can be either manic or depressive) are common. The estimated prevalence of Bipolar Disorder 1 ranges from 0.4-1.6%; different figures are mostly due to differences in the populations analysed and the definitions employed.

A manic episode is a period of abnormally elevated mood, accompanied by abnormal behavior that disrupts life, and includes, for example, flying suddenly from one idea to the next; rapid, “pressured,” and loud speech; increased energy, with hyperactivity and a decreased need for sleep; inflated self-image; excessive spending; hypersexuality; and/or substance abuse. Elevated mood can manifest itself as either euphoria or as irritability.

Many people with Bipolar Disorder 1 also suffer from episodes of depression. There may be a cycling between episodes of mania and depression. In between episodes of mania and depression, many people suffering from Bipolar Disorder 1 experience periods of remission or recovery which are essentially symptom free, and can live normal lives. There is therefore a genuine clinical benefit in a safe and effective therapy which can prevent the recurrence or relapse of Bipolar Disorder 1 in patients manifesting as episodes, for example manic, hypomanic, depressive or mixed episodes.

A minority of sufferers have rapid-cycling periods of manic and depressive episodes, with distinct periods of mania or depression four or more times within a year.

A mixed episode is characterized by the simultaneous occurrence of manic and depressive symptoms, or the fluctuation between manic and depressive symptoms within the same day.

Management of bipolar disorder patients includes both the treatment of acute manic/depressive episodes and the prevention of recurrent mood episodes. Lithium, valproate or atypical antipsychotics are usually first line treatment for acute mania episodes whereas haloperidol and carbamazepine are used as second line alternatives.

Manic episodes usually begin abruptly and last for between 2 weeks and 4-5 months (median duration about 4 months). Depressive episodes tend to last longer (median duration about 6 months). Recovery may or may not be complete between episodes. The pattern of remissions/recovery and relapses/recurrences is very variable, although remissions tend to get shorter as time goes on and depressions to become commoner and longer lasting. A return of symptoms at a subsyndromal level (sometimes referred to as “roughening”) may give an indication of a relapse/recurrence. Whilst acute treatment/management of an episode is important, the prevention or prophylaxis of relapse or of recurrence of further episodes is crucial for long-term (for example for at least 2 years following an acute episode) management of the disorder. Maintenance treatment is the use of a drug over a prolonged period of time. Such maintenance treatment can be used, for example, to reduce the severity of each acute episode as and when it arises, or to reduce the frequency of an episode associated with Bipolar Disorder 1.

Over the past decades, several alternative therapies for bipolar disease became available, particularly for the treatment of manic episodes. However, a significant proportion of patients remains refractory to these agents or cannot tolerate the adverse events (AE) (e.g. kidney and thyroid disorders associated with lithium therapy, testosterone increase associated with valproate or rash in patients starting lamotrigine or carbamazepine treatment).

Eslicarbazepine acetate ((S)-10-acetoxy-10,11-dihydro-5H-dibenz[b,f]azepine-5-carboxamide) is a potent voltage-gated sodium channel blocker described, e.g., in WO-A-97/02250, WO-A-2006/121363, WO-A-2007/094694, WO-A-2008/088233, WO-A-2009/054743, WO-A-2011/014084, WO-A-2011/031176, and WO-A-2012/091593, the contents of which applications are incorporated herein by reference. Eslicarbazepine acetate has been approved by the European Medicines Agency (EMA) for adjunctive therapy for partial-onset seizures, with or without secondary generalization, in adults with epilepsy.

Eslicarbazepine acetate is one of several drugs in the carboxamide dibenzazepine family. Other drugs in this family include oxcarbazepine (10,11-dihydro-10-oxo-5H-dibenz[b,f]azepine-5-carboxamide, OXC) and carbamazepine (5H-dibenzo[b,f]azepine-5-carboxamide, CBZ). Eslicarbazepine acetate gives reduced production of toxic metabolites compared to carbamazepine, leading to a better tolerability profile.

Eslicarbazepine acetate is metabolized in vivo in humans to the active metabolite, eslicarbazepine ((S)-10-hydroxy-10,11-dihydro-5H-dibenz[b,f]azepine-5-carboxamide), with R-licarbazepine and OXC as minor metabolites. Details can be found in “The Treatment of Epilepsy”, 3^{rd} edition, eds. Shorvon, Perucca & Engel, Chapter 38 (Almeida, L et al) (2009), the contents of which are incorporated herein by reference. Oxcarbazepine is also known to be metabolized in vivo in humans to eslicarbazepine and R-licarbazepine in a ratio of approximately 4:1.

Given the problems reported with existing therapies for treating and preventing the recurrence/relapse of Bipolar Disorder 1 and its associated episodes, there exists a need for the development of new treatments with better efficacy, safety and tolerability profiles. There also exists a need for therapies which are safe and effective in treating an acute manic or mixed episode in a patient suffering from Bipolar Disorder 1 and/or in preventing recurrence/relapse of the disorder.

### SUMMARY OF THE INVENTION

It has surprisingly been found that eslicarbazepine acetate and eslicarbazepine can be used to prevent recurrence/relapse of Bipolar Disorder 1 and the mood episodes associated therewith.

Accordingly, the present invention provides a drug selected from eslicarbazepine acetate and eslicarbazepine, for use in preventing the recurrence/relapse of Bipolar Disorder 1 and/or one or more episodes associated with Bipolar Disorder 1 in a human patient.

The present invention also provides a pharmaceutical composition for use in preventing the recurrence/relapse of Bipolar Disorder 1 and/or one or more episodes associated with Bipolar Disorder 1 as defined herein, in a patient as defined herein, which pharmaceutical composition comprises a pharmaceutically acceptable carrier and, as active principle, a drug as defined herein.

The present invention also provides use of a drug as defined herein, or a pharmaceutical composition as defined herein, in the manufacture of a medicament for use in preventing recurrence/relapse of Bipolar Disorder 1 and/or one or more episodes associated with Bipolar Disorder 1 as defined herein, in a patient as defined herein.

The present invention also provides a method of preventing the recurrence/relapse of Bipolar Disorder 1 and/or one or more episodes associated with Bipolar Disorder 1 as defined herein, in a patient as defined herein, which method comprises administering to said patient a safe and effective amount of a drug as defined herein, or a pharmaceutical composition as defined herein.

The present invention also provides a method of preventing the recurrence/relapse of Bipolar Disorder 1 and/or one or more episodes associated with Bipolar Disorder 1 as defined herein, which method comprises:

(a) selecting a patient as defined herein; and

(b) administering to said patient a safe and effective amount of a drug as defined herein, or a pharmaceutical composition as defined herein.

### DESCRIPTION OF THE FIGURES

FIG. 1 shows patient disposition for the clinical trials described in the Examples section.

FIG. 2 shows the relative change from baseline in total Young Mania Rating Scale score in study BIA-2093-203.

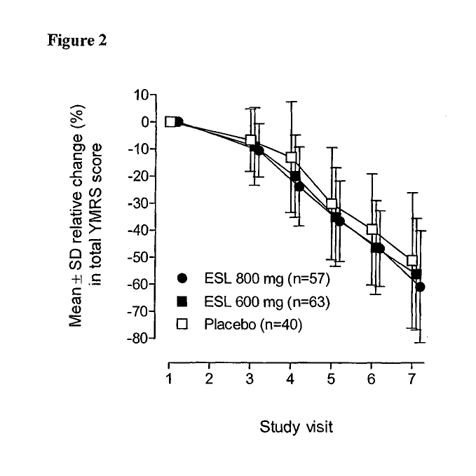

FIG. 3 shows the proportion of patients in full remission over the 3-week treatment period of study BIA-2093-203.

FIG. 4 shows the Highest and Lowest mood scores (patient diary card) in open-label and double-blind period (by weeks) of study BIA-2093-205 using a visual analogue scale.

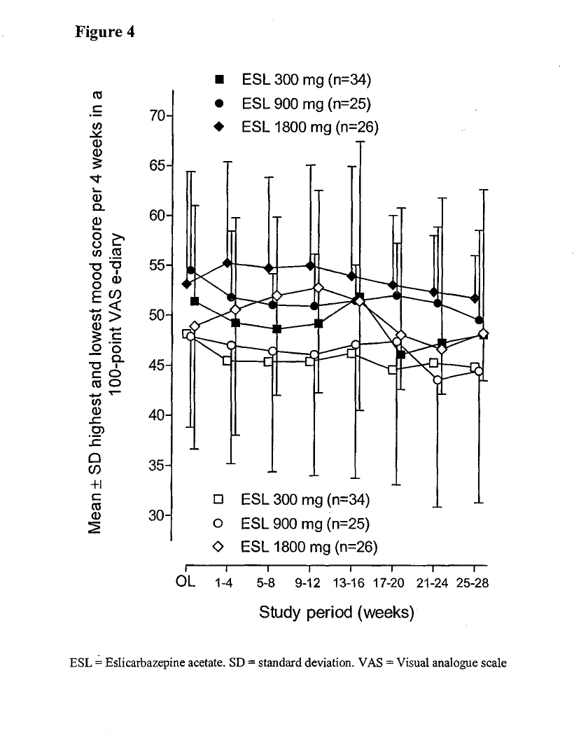

### DETAILED DESCRIPTION OF THE INVENTION

As used herein, the words “treatment” and “treating” are to be understood as embracing treatment and/or amelioration and/or prevention of or reduction in aggravation/worsening of symptoms of a disease or condition as well as treatment of the cause of the disease or condition, and may include reversing, reducing, or arresting the symptoms, clinical signs, and underlying pathology of a condition in a manner to improve or stabilise a subject's condition.

Reference to “prevention” and “preventing” a disease or condition embraces prophylaxis and/or inhibition of the disease or condition. The term “preventing” is art-recognized, and when used in relation to a condition, such as Bipolar Disorder 1 and its associated episodes, is well understood in the art, and includes administration of a drug and/or composition which reduces the frequency of, or delays the onset of, symptoms of a medical condition in a subject relative to a subject which does not receive the drug or composition.

In the present invention, a drug chosen from eslicarbazepine acetate and eslicarbazepine is used to prevent the recurrence/relapse of Bipolar Disorder 1 and/or one or more episodes associated with Bipolar Disorder 1.

Bipolar Disorder 1 manifests as mood episodes, so the drug chosen from eslicarbazepine acetate and eslicarbazepine is typically used to prevent the recurrence/relapse of the one or more mood episodes associated with Bipolar Disorder 1. Typical episodes include manic with or without psychotic symptoms, hypomanic, cyclothymic, euthymic, psychotic, euphoric, dysphoric, mixed and/or depressive with or without psychotic symptoms episodes. Depressive episodes can be mild, moderate or severe. Preferably, the drug chosen from eslicarbazepine acetate and eslicarbazepine is used to prevent the recurrence/relapse of one or more manic, hypomanic, mixed and/or depressive episodes associated with Bipolar Disorder 1.

Thus, prevention of the recurrence or relapse of Bipolar Disorder 1 includes, for example, delaying the onset (prolonging the time between episodes) or reducing the number (incidence), frequency, severity or duration of one or more of the typical episodes defined above in a treated population versus a control population untreated with eslicarbazepine or eslicarbazepine acetate, e.g., by a statistically and/or clinically significant amount.

It is of clinical benefit to prevent recurrence/relapse of Bipolar Disorder 1 and/or one or more episodes associated with Bipolar Disorder for as long a period of time as possible to ensure the best quality of life for the patient. Remission may be defined as absence or minimal symptoms of one or more episodes for at least one week, typically without worsening of symptoms of the opposite pole, for example reduced symptoms of depression does not involve worsening of manic symptoms. Thus, typically, prevention of recurrence/relapse refers to prevention of recurrence for a particular period of time, typically a period of at least one week, or one month or more, two months or more, or three months or more, or 6 months or more, or 9 months or more, or 12 months or more, or 15 months or more, or 18 months or more, or 21 months or more, or 24 months or more. Prevention over a longer period of time may referred to as “recovery” or “sustained remission”.

Reference to “recurrence” or “relapse” indicates that a patient has previously suffered from Bipolar Disorder 1 and/or one or more episodes associated with Bipolar Disorder 1, as defined above. Thus, typically, the patient has previously experienced one or more manic, hypomanic, mixed and/or depressive episodes, or one or more manic, hypomanic and/or mixed episodes, or one or more manic and/or mixed episodes, or one or more manic episodes. Typically, the patient has been diagnosed as suffering from Bipolar Disorder 1 in accordance with DSM-IV, the entirety of which is incorporated herein by reference. Typically, the patient has previously experienced one or more episodes associated with Bipolar Disorder 1 as defined herein and the drug for use in the present invention acts to prevent further such episodes of any duration. A patient “reoccurs” or “relapses” when, having experienced a period of remission or recovery, the patient experiences one or more symptoms of a mood episode. A return of symptoms at a subsyndromal level (sometimes referred to as “roughening”) may precede a relapse/recurrence, and trigger the need for administration of a drug according to the invention.

Typically, a patient has experienced one or more episodes associated with Bipolar Disorder 1, as defined above, and essentially symptom free periods (“remission” or “recovery”) between episodes. The administration of a drug selected from eslicarbazepine and eslicarbazepine acetate typically extends those symptom free periods for as long as possible, for example for a particular period of time, as defined above.

In certain embodiments, the patient suffers from rapid-cycling Bipolar Disorder 1.

A typical clinical situation presented in the treatment of Bipolar Disorder 1 is a patient suffering from an acute episode, typically a manic, hypomanic or mixed episode, usually a manic or mixed episode. The task of the clinician is first to address the acute episode, and also to set up a regime to prevent recurrence/relapse of Bipolar Disorder 1 and/or the episodes associated therewith for as long a time as is possible.

Typically, therefore, a patient treated in accordance with the present invention has previously been treated for Bipolar Disorder 1 and/or one or more episodes associated with Bipolar Disorder 1. Preferably, the patient has previously been treated for Bipolar Disorder 1 and/or one or more episodes associated with Bipolar Disorder 1 successfully, i.e. the patient treated in accordance with the present invention is typically no longer suffering from Bipolar Disorder 1 and/or one or more episodes associated with Bipolar Disorder 1, i.e. the patient is in remission or recovery. Typically, this previous treatment involves administering one or more therapeutic agents which are effective in treating Bipolar Disorder 1 and/or episodes associated with Bipolar Disorder as defined above. This previous treatment may also involve convulsant therapy, such as electroconvulsant therapy. Typically, the patient has previously been treated for Bipolar Disorder 1 and/or one or more episodes associated with Bipolar Disorder 1 using one or more therapeutic agents for the treatment of Bipolar Disorder 1 and/or episodes associated with Bipolar Disorder 1, as defined above. In certain embodiments, the one or more therapeutic agents include eslicarbazepine acetate and/or eslicarbazepine, typically eslicarbazepine acetate. In other embodiments, the one or more therapeutic agents are other than eslicarbazepine acetate and/or eslicarbazepine and may, for instance, include lithium, anticonvulsants such as (sodium) valproate, carbamazepine, and lamotrigine, and antipsychotics such as risperidone, olanzapine and aripiprazole.

In certain embodiments the patient is unresponsive to or does not tolerate treatment with another therapeutic agent such as lithium, (sodium) valproate, carbamazepine, lamotrigine, risperidone, olanzapine and/or aripiprazole. In certain embodiments, treatment with another therapeutic agent such as lithium, (sodium) valproate, carbamazepine, lamotrigine, risperidone, olanzapine and/or aripiprazole is contra-indicated.

In certain embodiments, the patient has previously been treated for Bipolar Disorder 1 and/or one or more episodes associated with Bipolar Disorder 1 using eslicarbazepine acetate and/or eslicarbazepine, and eslicarbazepine acetate and/or eslicarbazepine is then also used to prevent recurrence/relapse thereof. The present invention therefore also provides a drug selected from eslicarbazepine acetate and eslicarbazepine, for use in treating Bipolar Disorder 1 and/or one or more episodes associated with Bipolar Disorder 1 and preventing the recurrence/relapse of Bipolar Disorder 1 and/or one or more episodes associated with Bipolar Disorder 1 in a human patient. In such embodiments, the dosage of the drug administered to treat and prevent recurrence/relapse of Bipolar Disorder 1 and/or one or more episodes associated with Bipolar Disorder 1 may be the same or different.

The drug is particularly useful in the continuous treatment of patients who are susceptible to recurrence/relapse of Bipolar Disorder 1 and/or one or more episodes associated with Bipolar Disorder 1, as defined herein. The drug as defined above can be used as maintenance therapy to prevent the recurrence/relapse of episodes associated with Bipolar Disorder I, and/or to improve the patient's condition.

The severity of Bipolar Disorder 1, in particular the manic and manic-type episodes associated therewith can be measured by reference to one or more standard indices. The Young Mania Rating Scale (YMRS) (Young R C, Biggs J T, Ziegler V E, Meyer D A. A rating scale for mania: reliability, validity and sensitivity. The British Journal of Psychiatry: the journal of mental science. 1978; 133:429-35. Epub 1978 Nov. 1.) and Clinical Global Impressions Scale for use in bipolar illness (CGI-BP) (Spearing M K, Post R M, Leverich G S, Brandt D, Nolen W. Modification of the Clinical Global Impressions (CGI) Scale for use in bipolar illness (BP): the CGI-BP. Psychiatry Research. 1997; 73(3):159-71. Epub 1998 Mar. 3.) are widely used measures of mania and bipolar disorder which are sensitive to drug effects (Spearing et al, and Note for guidance on clinical investigation of medicinal products for the treatment and prevention of bipolar disorder, CPMP/EWP/567/98 (2001).).

The YMRS score is the sum of 11 scoring items, ranging from 0 to 60 points; higher scores indicate higher mania symptomatology.

The CGI-BP scale measures severity and treatment-related improvement in mania, depression and overall illness categories. It comprises 3 scales: severity of illness (1—normal to 7—very severely ill, in which 3—mildly ill is typically used as cut-off value for statistical analysis), change from preceding phase and worst phase (1—very much improved to 7—very much worse, and 8—not applicable).

The Montgomery-Åsberg Depression Rating Scale (MADRS) (Montgomery S A, Asberg M. A new depression scale designed to be sensitive to change. The British Journal of Psychiatry: the journal of mental science. 1979; 134:382-9. Epub 1979/04/01) is a widely accepted measure of depression designed to be sensitive to change (Note for guidance on clinical investigation of medicinal products for the treatment and prevention of bipolar disorder, CPMP/EWP/567/98 (2001)). It is calculated as the sum of 10 scoring items, ranging from 0 to 60 points: higher scores indicate higher depression symptomatology.

Prevention of recurrence/relapse of Bipolar Disorder 1 and/or one or more episodes associated with Bipolar Disorder 1 can be signified by no worsening in the CGI-BP scale for a patient, and or a YMRS score of less than 15. Typically, prevention of recurrence/relapse of one or more manic, hypomanic or mixed episodes is signified by no worsening in the CGI-BP scale for a patient, and or a YMRS score of less than 15.

Typically, the treatment of the present invention prevents recurrence/relapse of Bipolar Disorder 1 and/or one or more episodes associated with Bipolar Disorder 1 as determined by the YMRS, CGI-BP and/or MADRS scales, preferably the YMRS and/or CGI-BP scales.

Typically, the treatment of the present invention results in no worsening in the CGI-BP scale for the patient treated for as long as possible, for example for the particular period of time as defined above.

Typically, the treatment of the present invention results in a YMRS score of less than 15 being maintained for as long as possible, for example for the particular period of time as defined above.

Prevention of recurrence/relapse of Bipolar Disorder 1 and/or one or more episodes associated with Bipolar Disorder 1 can be signified by a MADRS score of less than 18. Typically, prevention of recurrence/relapse of one or more depressive episodes is signified by a MADRS score of less than 18.

Typically, the treatment of the present invention results in a MADRS score of less than 18 for as long as possible, for example for the particular period of time as defined above.

Typically, the patient treated in accordance with the present invention is not suffering from Bipolar Disorder 1 or an episode associated with Bipolar Disorder 1 as defined herein. Typically, the patient treated in accordance with the present invention is not suffering from an episode associated with Bipolar Disorder 1 as defined herein, preferably a manic or mixed episode. Typically, the patient treated in accordance with the present invention is not suffering from Bipolar Disorder 1 or an episode associated with Bipolar Disorder 1 as defined herein as determined by the YMRS, CGI-BP and/or MADRS scales.

Typically, the patient treated in accordance with the present invention is Caucasian.

In some embodiments, the patient treated in accordance with the present invention is at least 18 years of age. The patient treated in accordance with the present invention may be at least 16 years of age. In some embodiments, the patient treated in accordance with the present invention is less than 18 years of age.

Typically, the patient treated in accordance with the present invention is susceptible to Bipolar Disorder 1 and/or one or more episodes associated with Bipolar Disorder 1, or is susceptible to relapse or recurrence of one or more episodes associated with Bipolar Disorder 1. Patients susceptible to Bipolar Disorder 1 and/or one or more episodes associated with Bipolar Disorder 1 will typically have been diagnosed as suffering from Bipolar Disorder 1 and/or one or more episodes associated with Bipolar Disorder 1, preferably according to the DSM-IV criteria.

Patients susceptible to Bipolar Disorder 1 and/or one or more episodes associated with Bipolar Disorder 1 may have a family history of Bipolar Disorder 1 and/or other mood, affective or behavioral disorders and/or may already have experienced a mood, affective or behavioural disorder other than Bipolar Disorder 1.

Patients susceptible to relapse or recurrence of one or more episodes associated with Bipolar Disorder 1 may experience roughening.

Typically, treatment in accordance with the present invention improves the mood of the patient. Mood in treated patients can be self-evaluated, for instance using a 100-point visual analogue scale using, e.g., DiaryPRO™ software (Invivodata®, Inc.) implemented on a touch-screen device (Palm™ OS). Mood is typically assessed by reference to the reported highest and/or lowest mood states for a patient. Thus, typically, treatment in accordance with the present invention improves the highest and/or lowest mood of the patient. Typically, treatment in accordance with the present invention improves the average mood of the patient.

In some embodiments, the drug is eslicarbazepine acetate. In some embodiments, the drug is eslicarbazepine.

The drug may also be used for treating partial onset seizures, for example in patients with epilepsy who are also susceptible to or suffering from Bipolar Disorder 1 and/or (relapse or recurrence of) one or more episodes associated therewith. Therefore the patient may be suffering from partial onset seizures and/or epilepsy.

The drugs for use in the present invention may be administered as monotherapy treatment for the indication or with other drug(s) as adjunct therapy for the indication, as described in more detail below. In the case of adjunct therapy, the drugs for use in the present invention may be administered simultaneously, separately or sequentially with the other drug(s), for example in fixed dose combination or in separate doses.

The drugs for use in the present invention may be administered by any suitable route to provide a preventative therapeutic effect against Bipolar Disorder 1 and/or one or more episodes associated with Bipolar Disorder 1. Thus, they can be administered orally, for example as tablets, capsules, caplets, troches, lozenges, aqueous or oily suspensions, dispersible powders or granules. The drugs may also be administered parenterally, whether subcutaneously, intravenously, intramuscularly, intrasternally, transdermally or by infusion techniques. The drugs may also be administered as suppositories.

Typically, the drugs are for oral administration.

In one embodiment, the drugs are administered as a tablet or capsule.

In another embodiment, the drugs are administered as a suspension. This embodiment is explained further in WO-A-2011/031176, the content of which is incorporated herein by reference.

In a further embodiment, the drugs are administered as a granule formulation. This embodiment is explained further in WO-A-2012/091593, the content of which is incorporated herein by reference.

The drugs for use in the present invention may be administered once a day, or more than once a day, for example two, three or four times a day. Typically, the drugs are for once daily administration.

The drugs may be administered using a titration regime, starting on a lower dosage and increasing the dosage over time to the therapeutic dosage. For example, patients may start taking 400 mg once daily (QD) and titrate up in 400 mg steps until they are taking 800 mg or 1200 mg QD according to clinical response, or may start taking 800 mg once daily (QD) and titrate up in 800 mg steps until they are taking 1600 mg or 2400 mg QD according to clinical response, or may start taking 600 mg QD and titrate up in 600 mg steps until they are taking 1200 mg or 1800 mg according to clinical response. Titration may take place over several days or several weeks. For example, patients showing no improvement of symptoms over two, three, four, five days or over a week on one dosage may increase that dosage.

Dosages will vary depending on, e.g., the individual, the mode and frequency of administration, and the nature and severity of the condition to be treated. A clinician having ordinary skill in the art can readily determine and prescribe the effective amount required.

Typical doses for a patient will range from 1 mg per kilogram to 50 mg per kilogram of body weight per day. A typical daily oral dose of the drugs is from 100 mg to 4800 mg per day, preferably from 200 mg to 2400 mg per day, more preferably from 300 to 1800 mg per day. Examples of daily oral doses of the drugs include 300 mg per day, 400 mg per day, 600 mg per day, 700 mg per day, 800 mg per day, 900 mg per day, 1000 mg per day, 1100 mg per day, 1200 mg per day, 1300 mg per day, 1400 mg per day, 1500 mg per day, 1600 mg per day, 1700 mg per day, 1800 mg per day, 1900 mg per day, 2000 mg per day, 2100 mg per day, 2200 mg per day, 2300 mg per day, 2400 mg per day, 2500 mg per day, 2600 mg per day, 2700 mg per day, 2800 mg per day, 2900 mg per day, and 3000 mg per day. Specific examples of daily oral doses of the drugs include 400 mg per day, 600 mg per day, 800 mg per day, 900 mg per day, 1200 mg per day, 1800 mg per day, and 2400 mg per day.

Eslicarbazepine acetate and eslicarbazepine may be administered as a monotherapy, or in combination with one or more therapeutic agents for the prevention of recurrence/relapse of Bipolar Disorder 1 and/or one or more episodes associated with Bipolar Disorder 1. Typically, the drug is administered as a monotherapy, or in combination with one or more therapeutic agents for the prevention of recurrence/relapse of Bipolar Disorder 1 and/or one or more episodes associated with Bipolar Disorder 1 which is other than quetiapine administered at a dosage of 400 mg per day. Preferably the drug is administered as a monotherapy, or in combination with one or more therapeutic agents for the prevention of recurrence/relapse of Bipolar Disorder 1 and/or one or more episodes associated with Bipolar Disorder 1 which is other than quetiapine.

Typically, the present invention provides a drug selected from eslicarbazepine acetate and eslicarbazepine, for use in preventing the recurrence/relapse of Bipolar Disorder 1 and/or one or more episodes associated with Bipolar Disorder 1 in a human patient who is not receiving quetiapine administered at a dosage of 400 mg per day and/or levocetirizine administered at 5 mg per day.

Preferably, the present invention provides a drug selected from eslicarbazepine acetate and eslicarbazepine, for use in preventing the recurrence/relapse of Bipolar Disorder 1 and/or one or more episodes associated with Bipolar Disorder 1 in a human patient who is not receiving quetiapine and/or levocetirizine.

Suitable therapeutic agents for use in combination with eslicarbazepine acetate and eslicarbazepine include lithium, anticonvulsants such as sodium valproate, carbamazepine, and lamotrigine, and antipsychotics such as risperidone, olanzapine and aripiprazole.

The present invention also provides a pharmaceutical composition for use in preventing the recurrence/relapse of Bipolar Disorder 1 and/or one or more episodes associated with Bipolar Disorder 1 as defined herein, in a patient as defined herein, which pharmaceutical composition comprises a pharmaceutically acceptable carrier and, as active principle, a drug as defined herein.

Eslicarbazepine acetate and eslicarbazepine are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, povidone, carboxymethylcellulose or polyvinyl pyrrolidone; disaggregating agents, e.g. starch, alginic acid, alginates, croscarmellose soldium or sodium starch glycolate; effervescing mixtures; dyestuffs; sweeteners; flavouring agents; wetting agents, such as lecithin, polysorbates, laurylsulphates; and, in general, non toxic and pharmacologically inactive substances used in pharmaceutical formulations. Such pharmaceutical preparations may be manufactured in any known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, xanthan gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, a wetting agent, for example polyoxyethylene stearate, an antimicrobial agent, such as methylparaben or propylparaben, and if desired, a suitable amount of lidocaine hydrochloride.

Solutions for injection or infusion may contain as a carrier, for example, sterile water or preferably they may be in the form of sterile, aqueous, isotonic saline solutions.

Typically, the pharmaceutical composition is in the form of a tablet, granule formulation (i.e. for sprinkling on or adding to food or beverage) or suspension. Suitable tablets are described in WO-A-2009/054743. Suitable granule formulations are described in WO-A-2012/091593. Suitable suspensions are described in WO-A-2011/031176.

The present invention also provides use of a drug as defined herein, or a pharmaceutical composition as defined herein, in the manufacture of a medicament for use in preventing recurrence/relapse of Bipolar Disorder 1 and/or one or more episodes associated with Bipolar Disorder 1 as defined herein, in a patient as defined herein.

The present invention also provides a method of preventing the recurrence/relapse of Bipolar Disorder 1 and/or one or more episodes associated with Bipolar Disorder 1 as defined herein, in a patient as defined herein, which method comprises administering to said patient a safe and effective amount of a drug as defined herein, or a pharmaceutical composition as defined herein.

The present invention also provides a method of preventing the recurrence/relapse of Bipolar Disorder 1 and/or one or more episodes associated with Bipolar Disorder 1 as defined herein, which method comprises:

(a) selecting a patient as defined herein; and

(b) administering to said patient a safe and effective amount of a drug as defined herein, or a pharmaceutical composition as defined herein.

The following non-limiting Examples illustrate the invention.

### Examples

Two 3-week multicentre, double-blind, randomised, placebo-controlled studies in acute mania (study BIA-2093-203: dose titrated by response, eslicarbazepine acetate (ESL) 600-1800 mg or 800-2400 mg, once-daily; study BIA-2093-204: fixed doses of 600, 1200 and 1800 mg, once-daily) were followed by a recurrence prevention study consisting of a 2-week open-label period (900 mg, once-daily) continued by a double-blind, parallel-group, fixed dose (300, 900 and 1800 mg, once-daily) period for a minimum of 6 months. The primary endpoint was change in Young Mania Rating Scale (YMRS) in studies BIA-2093-203 and BIA-2093-204, and the proportion of no worsening patients in the Clinical Global Impressions-Bipolar Version (CGI-BP) in study BIA-2093-205.

### Patients and Methods

### Study Design

Study BIA-2093-203 (EudraCT No 2005-002131-27) followed a multicentre, double-blind, randomised, parallel-group, placebo-controlled, dose-titration design.

This study was conducted at 23 centres across Europe. Patients were randomised (3:3:2) to one of the following treatment groups: (1) ESL starting with 800 mg once daily (QD) and up-titrated in 800 mg steps until 2400 mg QD (maximum dose) according to clinical response, (2) ESL starting with 600 mg QD and up-titrated in 600 mg steps until 1800 mg QD (maximum dose) according to clinical response, and (3) Placebo QD. Patients were followed for up to 3 weeks. The study schedule consisted of a screening visit (V1), randomisation visit (V2, Day 1), and subsequent visits to evaluate clinical response (V3, Day 4; V4, Day 7; V5, Day 10; V6, Day 14 and V7, Day 21). In patients showing no improvement of symptoms, the dose of study medication was increased every 3 days until the maximum doses were reached. If maximum doses showed no effect for 3 days, the patient was tapered off and switched to an open-label escape therapy with an established antimanic drug. At the end of the 3-week treatment period, patients who responded to treatment had the option of entering the recurrence prevention study (Study BIA-2093-205).

Study BIA-2093-204 (EudraCT No 2005-002133-13) followed a multicentre, double-blind, randomised, parallel-group, placebo-controlled, fixed multiple dose design. This study was conducted at 25 study centres in Europe, South Africa and South America. Patients were randomised (1:1:1:1) to one of the following treatment groups: (1) ESL 1800 mg QD, (2) ESL 1200 mg QD, (3) ESL 600 mg QD, and (4) Placebo QD. The visit schedule in study BIA-2093-204 was similar to that of study BIA-2093-203. Patients who showed no improvement of symptoms by Day 10 were switched to open-label escape therapy with an established antimanic drug. Patients also had the option of entering Study BIA-2093-205.

Study BIA-2093-205 (EudraCT No 2005-002134-35) was a recurrence prevention study designed as a continuation of studies BIA-2093-203 and BIA-2093-204, and comprised two sequential parts. Part I followed an open-label design in which all participants received treatment with ESL 900 mg QD for 2 weeks. Part II followed a double-blind, parallel-group, fixed multiple dose design in which participants were randomly assigned (1:1:1) to one of the following treatment groups: (1) ESL 1800 mg QD, (2) ESL 900 mg QD, and (3) ESL 300 mg QD. The evaluations at the end of the 3-week treatment period in studies BIA-2093-203 and BIA-2093-204 served as admission procedure (V1) for study BIA-2093-205. Patients stable in remission continued double-blind therapy until approximately 6 months after the last patient entered Part II of Study BIA-2093-205. The occurrence of a new manic/depressive episode was considered a treatment failure, and the patient was discontinued from the study.

All the studies were conducted in accordance with local regulations, the ethical principles derived from the Helsinki Declaration and the Good Clinical Practice recommendations. The pertinent Ethics Committees and regulatory authorities approved the study protocol. The subjects provided their written informed consent prior to entering the study.

### Study Population

Studies BIA-2093-203 and BIA-2093-204 enrolled patients with ages ≧18 years, currently displaying an acute manic (including mixed) episode and with a documented diagnosis of bipolar I disorder according to the DSM-IV criteria (2). Eligible patients should have a Young Mania Rating Scale (YMRS) total score ≧20, with symptoms of the current manic episode starting within two weeks prior to randomization. Patients were excluded if they had history of schizophrenia or schizoaffective disorder, psychotic features or rapid cycling. Patients were also excluded if they were treated with carbamazepine, oxcarbazepine or a depot-neuroleptic. Previous treatment with ESL or history of unresponsiveness, intolerance or hypersensitivity to other dibenzazepine compounds (carbamazepine, oxcarbazepine or licarbazepine) also constituted criteria for exclusion. Patients with clinical relevant risk of harm to self or others and history of substance abuse were also excluded. Women were excluded if they were pregnant or breast-feeding, or if of childbearing potential they were unable to use double-barrier contraception.

The recurrence prevention study (BIA-2093-205) enrolled patients that completed the acute phase studies (BIA-2093-203 and BIA-2093-204) and responded to the treatment. Patients were excluded from Study BIA-2093-205 if any clinical relevant disorder arose at the time of inclusion.

### Concomitant Medications

Benzodiazepines were allowed in all studies. Prohibited bipolar disorder preventive medication included antidepressants, antipsychotics, antiparkinsonians, anxiolytics, monoamine oxidase inhibitors and other centrally acting drugs. Patients taking these medications had to be washed out for at least 2 days prior to randomisation, in studies BIA-2093-203 and BIA-2093-204; only patients with lithium plasma levels <0.5 mmol/L or valproic acid plasma levels <50 mg/L were admitted to study participation.

### Assessments

### Appropriateness of Measurements:

The following validated instruments were used: YMRS (20), the Clinical Global Impressions-Bipolar Version (CGI-BP) scale (21) and the Montgomery-Åsberg Depression Rating Scale (MADRS) (22). The YMRS score is the sum of 11 scoring items, ranging from 0 to 60 points; higher scores indicate higher mania symptomatology (20). The CGI-BP scale measures severity and treatment-related improvement in mania, depression and overall illness categories. It comprises 3 scales: severity of illness (1—normal to 7—very severely ill, in which 3—mildly ill was used as cut-off value for statistical analysis), change from preceding phase and worst phase (1—very much improved to 7—very much worse, and 8—not applicable) (21). The MADRS score is calculated as the sum of the 10 scoring items, ranging from 0 to 60 points: higher scores indicate higher depression symptomatology (22).

The YMRS and the CGI-BP are widely used measures of mania and bipolar disorder and they are sensitive to drug effects (21, 23). The MADRS is a widely accepted measure of depression designed to be sensitive to change (23).

### Efficacy

The primary efficacy endpoint for acute mania studies (BIA-2093-203 and BIA-2093-204) was the change in YMRS total score from baseline until the end of the 3-week treatment period. Investigators administered the YMRS at each visit. Secondary efficacy variables based on the YMRS score included responder rate (proportion of patients with ≧50% improvement or <12 points in the YMRS score), change in YMRS total score for each visit, proportion of patients in full remission (YMRS score <12), and time to full remission. The investigators completed CGI-BP in all visits and the test scores in the different categories were considered as secondary endpoints. Other secondary endpoints were the proportion of patients using benzodiazepines; proportion of patients remaining on treatment at the end of the study; and treatment retention time (study BIA-2093-203 only), defined as the time to withdrawal due to lack of efficacy or adverse events (AEs).

For the recurrence prevention study (BIA-2093-205), the primary efficacy endpoint was the proportion of patients who showed no worsening in the CGI-BP scale over Part II of the study. If the patient had—in change from preceding phase scale—a score of 5, 6, or 7 in any of 3 categories (mania, depression, or overall bipolar disorder), the illness was considered to have worsened. Secondary efficacy variables included: proportion of patients developing manic symptomatology (YMRS score ≧15); proportion of patients developing depressive symptomatology (MADRS score ≧18); the highest and lowest mood state, collected in the patient diary card; and time to withdrawal. All efficacy assessments were evaluated at each study visit.

During Study BIA-2093-205, patients used an electronic diary to document their mood stability (in terms of feeling depressive or manic). Mood was self-evaluated in a 100-point visual analogue scale using the DiaryPRO™ software (Invivodata®, Inc.) implemented on a touch-screen device (Palm™ OS). Patients completed their training in the e-diary use in V1 and were assisted by the study staff when necessary. Data were collected and transferred from the patient's home to the study server on a daily basis. Furthermore, secure web reports were generated based on these data, allowing trial staff to give feedback to subjects (at study visits) regarding their compliance.

### Safety

Medical history and demographic information was obtained at the screening visit. A complete physical and neurological examination was performed at screening, V7 or early discontinuation (EDV), V8 and post-study visit (PSV). Vital signs (pulse rate and blood pressure) were determined at every visit. A 12-lead ECG at rest was obtained at V1, V4, and V7, or at the EDV, and at PSV for patients who did not proceed to Study BIA-2093-205. Blood samples for biochemistry, haematology and coagulation were taken at screening, V4, V6 and V7 or EDV of studies BIA-2093-203 and BIA-2093-204; for patients that did not proceed to study BIA-2093-205, samples were also collected at V8 or PSV. Clinically significant abnormalities in laboratory tests, vital signs or 12-lead ECG were considered as AEs.

Safety was evaluated based on the occurrence of AEs. AEs were documented by the investigator with reference to intensity, dates of occurrence and resolution, outcome and relation to the treatment (causality). Furthermore, each AE was classified as being serious or non-serious. All patients with AEs were followed until their resolution. AEs were coded according to the Medical Dictionary for Regulatory Activities (MedDRA).

### Data Sets

Three study populations were defined for the statistical analyses: safety, intent to treat (ITT), and per protocol (PP). The safety population consisted of all patients who received at least one dose of investigational product. The ITT population consisted of all randomised patients who had at least one post-baseline efficacy assessment. The PP population consisted of all patients who complied with the study protocol without major deviations.

### Statistical Methods

Demographic data and other baseline characteristics were summarised by treatment group using descriptive statistics.

Proportions of patients were compared between treatment groups using a Cochran-Mantel-Haenszel (CMH) test stratified by region. For studies BIA-2093-203 and BIA-2093-204, the variables to be analysed were the proportion of patients in full remission, response (responder rate), use of benzodiazepines, and remaining on treatment. For study BIA-2093-205 the variables analysed were the proportion of patients showing no worsening according to CGI-BP, developing manic/depressive symptomatology, and withdrawing because of AEs. In all analyses, if the treatment effect was significant (p<0.05) pairwise comparisons were to be performed to assess the differences.

Changes in total YMRS, CGI-BP and MADRS scores were tested by analysis of covariance (ANCOVA) using baseline score value, region, treatment and region-by-treatment interaction as covariates; Dunnett's multiple comparison procedure was used for the comparison of the treatment means. ANCOVA was used to evaluate the change in YMRS score from baseline to the end of the 3-week treatment period, the CGI-BP score at the end of the 3-week treatment period, the MADRS score at the end of the 3-week treatment period, and the highest and lowest mood states (the mean highest and lowest score for each patient was calculated for intervals of 28 calendar days).

Survival analysis was performed using log-rank test/Kaplan-Meier and Cox proportional hazards model. For studies BIA-2093-203 and BIA-2093-204, treatment group comparisons were performed using Kaplan-Meier survival curves; the endpoints analysed were the time to full remission and the treatment retention time (Study BIA-2093-203 only). For Study BIA-2093-205 the time to withdrawal was analysed by Cox proportional hazards model.

In all studies missing values were addressed using last observation carried forward (LOCF) for efficacy data, for both ITT and PP populations.

All statistical analyses were performed using SAS® software, version 8.2 (SAS Institute Inc., Cary, N.C., USA).

### Sample Size Calculation

Assuming a common standard deviation (SD) of 11 for change from baseline in YMRS total score, it was estimated that a sample size of 160 patients (60 patients in each ESL group and 40 in the placebo group in study BIA-2093-203; 40 patients per group in study BIA-2093-204) would be required to detect a difference of 6.4 points in the primary endpoint in comparison to placebo using a 2-sided t-test with a power of 80% and an alpha level of 0.05. Assuming a response rate (secondary efficacy endpoint) of 30% for the placebo group, it was also estimated that with 160 patients the test would be able to detect a difference of approximately 30% between the response rates of the ESL groups and the placebo group (24). Since Study BIA-2093-205 was an extension of studies BIA-2093-203 and BIA-2093-204, the number of patients who would enter the study could not be predicted and no sample size was formally calculated.

### Results

### Patient Disposition and Characteristics

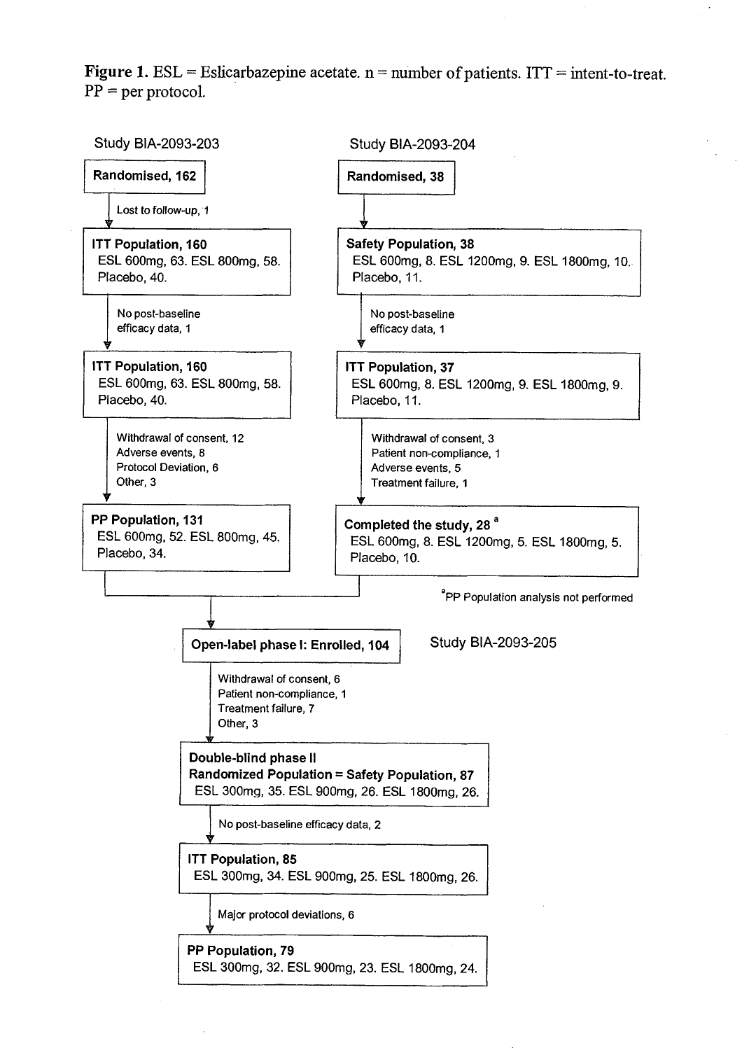

Patient disposition is displayed in FIG. 1 and demographic characteristics are shown table 1.

Study BIA-2093-203 was completed as planned: 161 patients received at least one dose of study medication and constituted the safety population. Similar percentages of patients in each treatment group discontinued from the study prematurely. The most common reason for discontinuation was withdrawal of consent. All patients in study BIA-2093-203 were Caucasian.

Study BIA-2093-204 was prematurely terminated due to patient recruitment difficulties; only 38 patients were randomised, and all of them received study medication. Most patients were Caucasian (84.2%).

Study BIA-2093-205 enrolled 104 patients in Part I, and 87 patients were randomised and received treatment in Part II. Overall, 35 (33.7%) patients completed 6 months of treatment. The ESL 1800 mg group contained the largest proportion of patients who prematurely discontinued (n=19, 73.1%), followed by the ESL 300 mg (n=19, 54.3%) and the ESL 900 mg (n=14, 53.8%) groups. The most common reason for premature discontinuation was treatment failure (n=22, 21.2%) with similar proportions between treatment groups. All but two patients were Caucasian.

For all the studies, no relevant differences existed between treatment groups in the use of prior and concomitant medications. The overall patient compliance to study medication was >95% in all studies.

### Efficacy

### Acute Mania Studies (BIA-2093-203 and BIA-2093-204)

### Primary Analyses

Table 2 displays the YMRS absolute change from baseline results.

In Study BIA-2093-203, the three treatment groups had similar YMRS scores (±SD) at baseline (placebo: 27.6±3.8; ESL 600-1800 mg: 27.0±5.1; ESL 800-2400 mg: 28.3±5.4). None of the treatment groups showed a reduction in YMRS total score as compared with placebo that was statistically significant.

For the ITT population of study BIA-2093-204, YMRS total scores decreased from baseline to the end of the 3-week treatment period for all treatment groups. This decrease was not dose related. Due to the reduced number of patients, the ANCOVA and the PP analysis were not performed.

### Recurrence Prevention Study (BIA-2093-205)

### Primary Analysis

The proportion of patients who showed no worsening according to the CGI-BP scale is presented in Table 2. In the ITT population, at least 50% of patients showed no worsening in all treatment groups. There was no statistically significant difference in effect based on the dosage of ESL administered (p=0.250). Results were similar in the PP population.

### Secondary Analyses

Proportion of Patients Who Developed Manic/Depressive Symptomatology:

For the ITT population, the proportion of patients with YMRS scores ≧15 (manic symptomatology) was of 8.8% for ESL 300 mg, 16.0% for ESL 900 mg, and 19.2% for ESL 1800 mg. The proportion of patients with MADRS scores ≧18 (depressive symptomatology) was of 14.7% for ESL 300 mg, 0.0% for ESL 900 mg, and 11.5% for ESL 1800 mg. Again, there was no statistically significant difference in effect based on the dosage of ESL administered (p=0.543 for YMRS and p=0.127 for MADRS). Results in the PP population were similar.

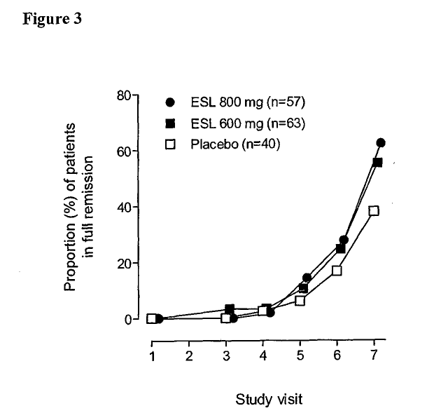

Highest and Lowest Mood State as Assessed by the Patient e-Diary (FIG. 3):

The mean (SD) highest mood scores for ESL 300 mg, 900 mg, and 1800 mg groups during the double-blind period in the ITT population were respectively 49.2 (11.0), 50.6 (6.4), and 55.1 (10.4). ANCOVA analysis (using as baseline the initial open-label period) revealed statistically significant differences between dosage groups for the ITT population at weeks 1-4 (p=0.006) and overall period (p=0.016). PP population presented similar results (p=0.004 at weeks 1-4 and p=0.009 in the overall period).

The mean (SD) overall lowest mood scores for the ESL 300 mg, 900 mg, and 1800 mg groups during the double-blind period in the ITT population were respectively 45.9 (10.4), 47.3 (12.4), and 51.1 (12.3). Results were similar for the PP population.

Time to Withdrawal:

Patients withdrew more often from the ESL 1800 mg group (73.1%), followed by the ESL 300 mg (52.9%) and the ESL 900 mg groups (52.0%). Patients from the ESL 1800 mg group had a shorter median time to withdrawal (109 days), followed by the ESL 300 mg (254 days) and the ESL 900 mg (295 days) groups. However, differences between the dosage groups were not statistically significant (p=0.097). Results were similar for the PP population (p=0.104).

### Safety

### Occurrence of AEs

Table 3 presents the incidence of AEs occurring per treatment group. AEs occurred more frequently in the ESL groups than in the placebo group. The overall frequency of AEs was similar in the ESL 600-1800 mg and ESL 800-2400 mg of study BIA-2093-203 groups whereas the ESL 1200 mg and ESL 1800 mg groups in study BIA-2093-204 had a higher frequency of AEs but with a limited number of patients. For all treatment groups, the most frequent types of AEs were nervous system and gastrointestinal disorders; these AEs also occurred more frequently in the ESL groups than in the placebo group.

In study BIA-2093-205, 42 AEs were reported in 20 patients (19.2%) during the open-label period. During the subsequent double-blind period, AEs incidence showed to be dose-dependent. Patients withdrew more frequently from the ESL 900 mg group (n=3, 11.5%), followed by the ESL 300 mg (n=3, 8.6%) and ESL 1800 mg groups (n=1, 3.8%).

Most AEs were mild or moderate in intensity and resolved by the end of the treatment period. In studies BIA-2093-203 and BIA-2093-204, the number of patients with serious AEs was small and comparable for all treatment groups. One placebo patient died following an ischemic stroke in the tapering-off period after V7. Other serious AEs included mania (n=2, ESL 800 mg; n=1, ESL 1800 mg; n=1, placebo; and n=1, ESL 600 mg), and 1 case of leukopenia and hyponatremia with ESL 600 mg.

In study BIA-2093-205, the number of patients with serious AEs was also small and comparable for all treatment groups. No deaths occurred. The serious AEs identified were: 2 cases of mania in the ESL 300 mg group; 1 case of anaemia, 1 case of oesophageal stenosis, 1 case of disease progression, and 1 case of pneumonia in the ESL 900 mg group; 1 case of disease progression, 1 case of bronchitis, and 1 case of depression in the ESL 1800 mg group. However, it is noteworthy that mania and depression should be considered normal progression of bipolar disorder (i.e. classified as treatment failure and not as AEs); the same applies for the AE “disease progression”.

In studies BIA-2093-203 and BIA-2093-204, the incidence of treatment-related AEs was relatively low (<35% of patients) and comparable between treatment groups, except for the possibly related AEs, which occurred more often in the ESL treatment groups than in the placebo group. For study BIA-2093-204 data on relationship to treatment are not available because a formal analysis of AEs was not performed. For the recurrence prevention study, the incidence of treatment-related AEs was also low (<25% of patient) and comparable between treatment groups.

### MADRS Score

There was an apparent improvement in MADRS scores under treatment with ESL as compared to placebo in study BIA-2093-203. However, the ANCOVA with treatment and region as fixed effects and baseline value as covariate showed no significant difference between the treatment groups at visit 7. Study BIA-2093-204 MADRS scores were only evaluated descriptively: for most groups the mean changes from baseline were small (except for reduced sleep and concentration difficulties score items).

### Discussion

The studies reported here tested the efficacy of ESL in acute mania (studies BIA-2093-203 and BIA-2093-204) and recurrence prevention (study BIA-2093-205) of bipolar disorder I. Overall, ESL showed a trend for efficacy, mainly in YMRS scores and remission rates. AEs with ESL showed to be mostly of mild to moderate intensity and consistent with previously reported observations.

In study BIA-2093-203, 162 patients were randomised, matching the calculated sample size (n=160). The study was therefore adequately powered. Patient enrolment was, however, lower than planned for studies BIA-2093-204 and BIA-2093-205.

Treatment groups from studies BIA-2093-203 and BIA-2093-205 were well matched with respect to demographic characteristics and medical conditions at baseline. The use of prior and concomitant medications was similar between the treatment groups for all studies. Also the patient compliance rates were very high and homogeneous, registering a minimum value of 95%.

In general, study BIA-2093-203 efficacy results were consistent, trending to an increase in efficacy across the dosage regimens from placebo to ESL 800-2400 mg group. ESL 800-2400 mg produced a greater reduction in YMRS total score than placebo (−14.2 vs −10.3) (p=0.0523). The absolute differences observed between the ESL treatment groups and placebo for the YMRS total score were smaller than those used for sample size calculation (4.0 for ESL 800 mg and 2.2 for ESL 600 mg versus 6.4 in the sample size calculation). Moreover, a significantly higher proportion of patients were in full remission at V7 in the ESL 800-2400 mg group when compared to the placebo group. The proportion of patients in V7 classified as normal (by CGI-BP severity of illness scale) increased when doses increased, showing significant differences between the ESL 800 mg group and placebo group for mania and overall bipolar illness. CGI-BP score changes for mania and overall bipolar illness indicate a significant improvement in patient symptomatology for the ESL 800 mg group (from preceding and worst phase) and for ESL 600 mg group (from worst phase only) when compared to placebo.

In study BIA-2093-204, the mean change in YMRS total scores from screening to V7 ranged from −11.3 in the ESL 1800 mg group to −17.7 in the placebo group. This apparent lower effectiveness of ESL 1800 mg in comparison to placebo may be justified by a higher YMRS score of ESL 1800 mg group at baseline and the use of LOCF. Most patients in all treatment groups were responders and were in full remission at the end of the 3-week treatment period (except for the ESL 1800 mg group: 33.3%). CGI-BP scores generally decreased for all groups and conditions over the course of the study.

Response rates to monotherapy in acute mania are referred in literature as rarely larger than 60% (25). Study BIA-2093-203 response rates are in the top limit of this reference, with ESL 800-2400 mg group presenting a response rate of 66.7%. Responders in study BIA-2093-204 reflect the low number of patients, but also the uncharacteristic population subset, with response rates ranging from 56% to 100%—with a placebo response rate of 90.9%.

Over the double-blind period of study BIA-2093-205, for highest mood scores, statistically significant differences were found between dosage groups—higher mood scores were associated with higher ESL doses—at weeks 1 through 4 and overall period.

In studies BIA-2093-203 and BIA-2093-204, AEs occurred more frequently in the ESL treatment groups than in the placebo group. The most frequent types of AEs were nervous system and gastrointestinal disorders, being in the vast majority cases of mild or moderate intensity and resolved by the end of the study. Severe intensity AEs were less frequent and included vomiting, agitation and mania. The number of patients with serious AEs was also small and comparable in all treatment groups. In study BIA-2093-205, ESL 1800 mg group caused more AEs than the lower dosage groups. Most AEs were of mild or moderate intensity and balanced across treatment groups. Psychiatric disorders were the most frequent AEs. The safety results are in line with previous clinical studies with ESL in epileptic patients that reported dizziness, somnolence, headache, nausea, diplopia and vertigo as the most common AEs. On these studies AEs leading to discontinuation included dizziness, abnormal coordination, and nausea.

### REFERENCES

2. American Psychiatric Association. Diagnostic and Statistical Manual of Mental Disorders. 4 ed. Washington D.C.: American Psychiatric Association; 2000.
20. Young R C, Biggs J T, Ziegler V E, Meyer D A. A rating scale for mania: reliability, validity and sensitivity. The British journal of psychiatry: the journal of mental science. 1978; 133:429-35. Epub 1978/11/01.
21. Spearing M K, Post R M, Leverich G S, Brandt D, Nolen W. Modification of the Clinical Global Impressions (CGI) Scale for use in bipolar illness (BP): the CGI-BP. Psychiatry research. 1997; 73(3):159-71. Epub 1998/03/03.
22. Montgomery S A, Asberg M. A new depression scale designed to be sensitive to change. The British journal of psychiatry: the journal of mental science. 1979; 134:382-9. Epub 1979/04/01.
23. Note for guidance on clinical investigation of medicinal products for the treatment and prevention of bipolar disorder, CPMP/EWP/567/98 (2001).
24. Vieta E, Mullen J, Brecher M, Paulsson B, Jones M. Quetiapine monotherapy for mania associated with bipolar disorder: combined analysis of two international, double-blind, randomised, placebo-controlled studies. Current medical research and opinion. 2005; 21(6):923-34. Epub 2005/06/23.

25. Goodwin G, Fleischhacker W, Arango C, Baumann P, Davidson M, de Hert M, et al. Advantages and disadvantages of combination treatment with antipsychotics ECNP Consensus Meeting, March 2008, Nice. European neuropsychopharmacology: the journal of the European College of Neuropsychopharmacology. 2009; 19(7):520-32. Epub 2009/05/05.

### TABLES

TABLE 1
Demographic characteristics of safety population by study and treatment
group
Study BIA-2093-203

| | Placebo | ESL 600 mg | ESL 800 mg |
|---|---|---|---|
| Characteristic | (N = 40) | (N = 63) | (N = 57) |
| Age in years: mean ± SD | 42.1 ± 13.2 | 43.1 ± 12.8 | 41.8 ± 13.0 |
| Male gender: n (%) | 22.0 (55.0) | 28.0 (43.8) | 30.0 (52.6) |
| Body weight in kg: mean ± SD | 81.8 ± 16.7 | 78.3 ± 17.6 | 76.0 ± 14.6 |
| BMI in kg/m²: mean ± SD | 27.8 ± 5.5 | 27.0 ± 4.8 | 26.1 ± 5.3 |

Study BIA-2093-204

| | Placebo | ESL 600 mg | ESL 1200 mg | ESL 1800 mg |
|---|---|---|---|---|
| | (N = 11) | (N = 8) | (N = 9) | (N = 10) |
| Age in years: mean ± SD | 44.5 ± 11.6 | 36.0 ± 12.0 | 40.3 ± 13.7 | 45.3 ± 18.2 |
| Male gender: n (%) | 3.0 (27.3) | 7.0 (87.5) | 0.0 (0.0) | 6.0 (60.0) |
| Body weight in kg: mean ± SD | 81.8 ± 16.7 | 76.5 ± 17.1 | 71.7 ± 13.3 | 74.9 ± 18.1 |
| BMI in kg/m²: mean ± SD | 2.0 ± 4.5^{a} | 26.8 ± 3.7 | 26.9 ± 5.0 | 27.5 ± 7.0 |

Study BIA-2093-205

| | ESL 300 mg | ESL 900 mg | ESL 1800 mg |
|---|---|---|---|
| | (n = 35) | (n = 26) | (n = 26) |
| Age in years: mean ± SD | 41.7 ± 13.0 | 43.1 ± 12.1 | 44.3 ± 11.6 |
| Male gender: n (%) | 19.0 (54.3) | 15.0 (57.7) | 15.0 (57.7) |
| Body weight in kg: mean ± SD | 78.4 ± 20.4^{b} | 73.8 ± 12.5 | 81.8 ± 12.8 |
| BMI in kg/m²: mean ± SD | 27.0 ± 5.4^{b} | 25.4 ± 4.9 | 27.7 ± 4.3 |

ESL = Eslicarbazepine acetate;
N = total number of patients;
n = number of patients;
BMI = body mass index;
SD = standard deviation.
^{a}Only 10 patients were included in this analysis;
^{b}Only 34 patients were included in this analysis.

TABLE 2
YMRS score absolute change in Studies BIA-2093-203 and BIA-2093-204, and patient
worsening status according to CGI-BP scale in Study BIA-2093-205 (intent-to-treat population).
YMRS score absolute change from V2 (1 Day) to V7 (21 Days)

| | Placebo | ESL 600 mg | ESL 800 mg |
|---|---|---|---|
| Study BIA-2093-203 | (n = 40) | (n = 63) | (n = 57) |
| Mean ± SD | −10.0 ± 9.7 | −12.2 ± 7.9 | −14.1 ± 8.9 |
| Median (range) | −12.0 (−31.0, 10.0) | −14.0 (−25.0, 14.0) | −16.0 (−33.0, 22.0) |

| | Placebo | ESL 600 mg | ESL 1200 mg | ESL 1800 mg |
|---|---|---|---|---|
| Study BIA-2093-204 | (n = 11) | (n = 8) | (n = 9) | (n = 9) |
| Mean ± SD | −17.7 ± 7.3 | −16.9 ± 2.8 | −16.7 ± 9.1 | −11.3 ± 10.9 |
| Median (range) | −17.0 (−33.0, −5) | −17.5 (−20.0, −12.0) | −15.0 (−35.0, −4.0) | −15.0 (−26.0, 7.0) |

Patient worsening status according to CGI-BP Scale^{a}

| | ESL 300 mg | ESL 900 mg | ESL 1800 mg |
|---|---|---|---|
| Study BIA-2093-205 | (n = 34) | (n = 25) | (n = 26) |
| No worsening n (%) | 26 (76.5) | 14 (56.0) | 16 (61.5) |
| Worsening n (%) | 6 (17.6) | 10 (40.0) | 6 (23.1) |

ESL = Eslicarbazepine acetate;
n = number of patients;
SD = standard deviation.
^{a}Subjects who had no worsening were included in the analysis only if they had at least 2 post-baseline assessments.

TABLE 3
Incidence of AEs occurring in at least 5% of patients or in at least 2 patients in any treatment
group (safety population).

| | |
|---|---|
| Studies BIA-2093-203 and BIA-2093-204 | Number (%) of patients |

| | Placebo | ESL 600 mg | ESL 800 mg | ESL 1200 mg | ESL 1800 mg |
|---|---|---|---|---|---|
| | (n = 51) | (n = 72) | (n = 57)^{a} | (n = 9)^{b} | (n = 10)^{b} |
| Any Adverse Events | 21 (41.2) | 38 (52.8) | 29 (50.9) | 9 (100.0) | 10 (100.0) |
| Dizziness | 5 (9.8) | 7 (9.7) | 5 (8.8) | 2 (22.2) | 4 (40.0) |
| Headache | 3 (5.9) | 8 (11.1) | 5 (8.8) | 5 (55.6) | 1 (10.0) |
| Vomiting | 3 (5.9) | 4 (5.6) | 2 (3.5) | 2 (22.2) | 2 (20.0) |
| Nausea | 2 (3.9) | 2 (2.8) | 4 (7.0) | 1 (11.1) | 3 (30.0) |
| Diarrhoea | 0 (0.0) | 8 (11.1) | 2 (3.5) | 0 (0.0) | 0 (0.0) |
| Somnolence | 1 (2.0) | 2 (2.8) | 1 (1.8) | 1 (11.1) | 3 (30.0) |
| Paraesthesia oral | 0 (0.0) | 0 (0.0) | 0 (0.0) | 1 (11.1) | 2 (20.0) |

| | |
|---|---|
| Study BIA-2093-205 | Number (%) of patients |

| | ESL 900 mg |
|---|---|
| Part I Open-Label Period | (n = 104) |
| Any Adverse Events^{c} | 20 (19.2) |

| | ESL 300 mg | ESL 900 mg | ESL 1800 mg |
|---|---|---|---|
| Part II Double-Blind Period | (n = 35) | (n = 26) | (n = 26) |
| Any Adverse Events | 14 (40.0) | 14 (53.8) | 18 (69.2) |
| Dizziness | 0 (0.0) | 0 (0.0) | 3 (11.5) |
| Nausea | 0 (0.0) | 0 (0.0) | 3 (11.5) |
| Bronchitis | 0 (0.0) | 1 (3.8) | 2 (7.7) |
| Blood creatine phosphokinase increased | 1 (2.9) | 0 (0.0) | 3 (11.5) |
| Aspartate amino-transferase increased | 0 (0.0) | 0 (0.0) | 2 (7.7) |
| Depression | 3 (8.6) | 3 (11.5) | 1 (3.8) |
| Agitation | 2 (5.7) | 0 (0.0) | 1 (3.8) |
| Sinus tachycardia | 2 (5.7) | 1 (3.8) | 0 (0.0) |
| Mania | 2 (5.7) | 0 (0.0) | 0 (0.0) |

ESL = Eslicarbazepine acetate;
n = number of patients.
^{a}This dosage was only present in study BIA-2093-203;
^{b}This dosage was only present in study BIA-2093-204;
^{c}In this period there were no adverse events occurring in at least 5% of patients or in at least 2 patients in any treatment group.

1. A drug selected from eslicarbazepine acetate and eslicarbazepine, for use in preventing the recurrence of Bipolar Disorder 1 and/or one or more episodes associated with Bipolar Disorder 1 in a human patient.

2. A drug for use according to claim 1, which is for use in preventing the recurrence of one or more episodes associated with Bipolar Disorder 1.

3. A drug for use according to claim 1 or 2, wherein the one or more episodes associated with Bipolar Disorder 1 are chosen from manic, hypomanic, mixed and/or depressive episodes.

4. A drug for use according to any one of the preceding claims, wherein the patient has previously been treated for Bipolar Disorder 1 and/or one or more episodes associated with Bipolar Disorder 1, as defined in claim 1 or 3.

5. A drug for use according to any one of the preceding claims, wherein the patient has previously received one or more therapeutic agents for the treatment of Bipolar Disorder 1 and/or one or more episodes associated with Bipolar Disorder 1.

6. A drug for use according to claim 5, wherein the one or more therapeutic agents include eslicarbazepine acetate and/or eslicarbazepine.

7. A drug for use according to claim 1, which is for use as a maintenance therapy.

8. A drug for use according to any one of the preceding claims, wherein the patient has previously experienced one or more episodes associated with Bipolar Disorder 1 as defined in claim 1 or 3.

9. A drug for use according to any one of the preceding claims, wherein the patient is Caucasian.

10. A drug for use according to any one of the preceding claims, wherein the drug is eslicarbazepine acetate.

11. A drug for use according to any one of the preceding claims, which is for use as a monotherapy.

12. A drug for use according to any one of the preceding claims, wherein the drug is administered at a daily dose selected from 400 mg, 600 mg, 800 mg, 900 mg, 1200 mg, 1800 mg, and 2400 mg.

13. A drug for use according to any one of claims 1 to 11, wherein the drug is administered at a starting dose of 400 mg per day and the dose is titrated upward in 400 mg steps according to clinical response.

14. A drug for use according to claim 13 wherein the dose is titrated upward to a dose of 1200 mg per day.

15. A drug for use according to any one of claims 1 to 11, wherein the drug is administered at a starting dose of 800 mg per day and the dose is titrated upward in 800 mg steps according to clinical response.

16. A drug for use according to claim 15 wherein the dose is titrated upward to a dose of 2400 mg per day.

17. A drug for use according to any one of claims 1 to 11, wherein the drug is administered at a starting dose of 600 mg per day and the dose is titrated upward in 600 mg steps according to clinical response.

18. A drug for use according to claim 17 wherein the dose is titrated upward to a dose of 1800 mg per day.

19. A drug for use according to any one of claims 1 to 10, wherein the patient receives one or more additional therapeutic agents for the prevention of recurrence of Bipolar Disorder 1 and/or one or more episodes associated with Bipolar Disorder 1.

20. A drug for use according to any one of the preceding claims, for oral administration.

21. A drug for use according to any one of the preceding claims, for once daily administration.

22. A pharmaceutical composition for use in preventing the recurrence of Bipolar Disorder 1 and/or one or more episodes associated with Bipolar Disorder 1 as defined in any one of claims 1 to 3, in a patient as defined in any one of claims 1, 4 to 6, 8 and 9, which pharmaceutical composition comprises a pharmaceutically acceptable carrier and, as active principle, a drug as defined in any one of claims 1 and 10 to 21.

23. A pharmaceutical composition for use according to claim 22, which is in the form of a tablet.

24. A pharmaceutical composition for use according to claim 22, which is in the form of a suspension.

25. A pharmaceutical composition for use according to claim 22, which is in the form of a granule formulation.

26. A method of preventing the recurrence of Bipolar Disorder 1 and/or one or more episodes associated with Bipolar Disorder 1 as defined in any one of claims 1 to 3, in a patient as defined in any one of claims 1, 4 to 6, 8 and 9, which method comprises administering to said patient a safe and effective amount of a drug as defined in any one of claims 1 and 10 to 21, or a pharmaceutical composition as defined in any one of claims 22 to 25.

27. A method of preventing the recurrence of Bipolar Disorder 1 and/or one or more episodes associated with Bipolar Disorder 1 as defined in any one of claims 1 to 3, which method comprises:
(a) selecting a patient as defined in any one of claims 1, 4 to 6, 8 and 9; and
(b) administering to said patient a safe and effective amount of a drug as defined in any one of claims 1 and 10 to 21, or a pharmaceutical composition as defined in any one of claims 22 to 25.
